(19) United States
(12) Patent Application Publication
(10) Pub. No.: US 2024/0336577 A1
(43) Pub. Date: Oct. 10, 2024

(54) PYRAZINE COMPOUND, AND METHOD OF PREPARATION AND USE THEREOF

(71) Applicant: SHENZHEN OLIVE BIOPHARMACEUTICALS CO., LTD, Shenzhen (CN)

(72) Inventors: Xifei YANG, Shenzhen (CN); Yongmei XIE, Shenzhen (CN); Shupeng LI, Shenzhen (CN)

(73) Assignee: SHENZHEN OLIVE BIOPHARMACEUTICALS CO., LTD, Shenzhen (CN)

(21) Appl. No.: 18/018,884

(22) PCT Filed: Jul. 30, 2021
(86) PCT No.: PCT/CN2021/109564

§ 371 Date: May 4, 2023

(30) Foreign Application Priority Data
Jul. 31, 2020 (CN) 202010759402.5
Jul. 31, 2020 (CN) 202010759405.9

(51) Int. Cl. C07D 241/12; A61K 31/4965; A61P 25/16; A61P 25/28

(52) U.S. Cl. CPC C07D 241/12; A61K 31/4965; A61P 25/16; A61P 25/28

(57) ABSTRACT

The present disclosure relates to use of a pyrazine compound in preparation of a drug. The drug can treat a neurodegenerative disease (ND) including Alzheimer's disease, Parkinson's disease, Huntington's disease, frontotemporal dementia (FTD), vascular dementia, HIV-related dementia, multiple sclerosis, progressive lateral sclerosis, Friedreich's ataxia, neuropathic pain, or glaucoma. The drug can further treat an inflammation, an oxidative damage, and a mitochondrial disorder-related disease.

[symbol]

### CROSS REFERENCE TO RELATED APPLICATION

This application is a national stage application of International Patent Application No. PCT/CN2021/109564, filed on Jul. 30, 2021, which claims priority of the Chinese Patent Application No. 202010759405.9 filed to the China National Intellectual Property Administration (CNIPA) on Friday, Jul. 31, 2020, entitled “PYRAZINE COMPOUND AND METHOD OF PREPARATION THEREOF”, and the Chinese Patent Application No. 202010759402.5 filed to the CNIPA on Friday, Jul. 31, 2020, entitled “USE OF PYRAZINE COMPOUND IN PREPARATION OF DRUG”, all of which are incorporated herein by reference in their entireties.

### TECHNICAL FIELD

The present disclosure relates to the field of medicines, in particular to a pyrazine compound, and a method of preparation and use thereof.

### BACKGROUND ART

Neurodegenerative disease (ND) is a chronic disease that leads to the progressive death of neurons, including Alzheimer's disease, Parkinson's disease, Huntington's disease, frontotemporal dementia (FTD), amyotrophic lateral sclerosis, and Friedreich's ataxia. The ND generally brings huge pain to patients and heavy burden to their families. As the population aging aggravates, the ND is expected to replace cancers as the second leading cause of human death by 2040. However, there is currently no drug in the world that can effectively treat the ND.

The pathology of ND is closely related to oxidative stress, mitochondrial dysfunction, Ca²⁺ influx, immune inflammation, autophagy and metal ions, such that the ND is a complex disease with multiple etiological factors. The traditional single-target and high-selectivity drug development strategy is not effective in the development of novel drugs for the ND. Natural molecules of traditional Chinese medicine have become a research hotspot of anti-ND drugs in recent years due to multiple therapeutic targets, less toxic and side effects, and desirable synergistic effect.

Diabetes mellitus (DM), as a lifelong metabolic disease caused by insulin secretion defect or insulin utilization disorder, is mainly characterized by hyperglycemia. With the improvement of residents' living standards and the changes in dietary structure, the DM has an annually-increasing incidence and a younger tendency. Diabetic nephropathy (DN) is one of the common chronic complications of the DM. The DN in the diabetic population has an incidence of about 20% to 40%, and about 50% c of DN patients may die of terminal renal failure in a later stage. Therefore, the DN is also a leading cause of death from chronic kidney diseases. The DN has extremely hidden, complex and diverse pathogenesis, and cannot be effectively treated in clinical practices.

Through long-term researches, the inventor has found a pyrazine compound that has a therapeutic effect on mitochondrial disorder-related diseases such as the ND and DM.

### SUMMARY

The present disclosure provides a pyrazine compound, a stereoisomer, a tautomer, and a pharmaceutically acceptable salt thereof. The pyrazine compound is a compound of formula I:

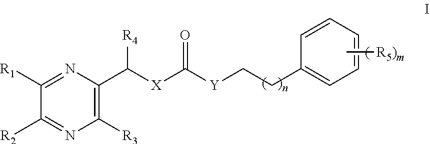

in which, X and Y each are independently selected from the group consisting of O, S, Se, and NR₆; R₁, R₂, R₃, R₄, R₅, and R₆ each are independently selected from the group consisting of H, deuterium, halogen, hydroxyl, amino, carboxyl, acylamino, ester, substituted or unsubstituted alkyl, deuterated alkyl, substituted or unsubstituted alkenyl, substituted or unsubstituted alkynyl, substituted or unsubstituted cycloalkyl, substituted or unsubstituted heterocyclyl, substituted or unsubstituted alkoxy, substituted or unsubstituted alkylcarboxyl, substituted or unsubstituted alkylester, -substituted or unsubstituted alkyl-OH, substituted or unsubstituted alkoxy, substituted or unsubstituted alkylamino, -substituted or unsubstituted alkyl-NH₂, substituted or unsubstituted aryl, substituted or unsubstituted heterocyclic aryl, substituted or unsubstituted carbonate, substituted or unsubstituted carbamate, -substituted or unsubstituted alkyl-acylamino, -substituted or unsubstitutedalkyl-aminocarboxylate, and a deuterated derivative thereof; and n is 0 to 6, m is 0 to 5.

In some embodiments, n may be 0, 1, 2, 3, 4, 5, or 6; and m may be 0, 1, 2, 3, 4, or 5.

In some embodiments, R₁, R₂, and R₃ each may be selected from the group consisting of methyl and deuterated methyl.

In some embodiments, R₄ may be selected from the group consisting of H and deuterium.

In some embodiments, the compound has a structure shown in formula II:

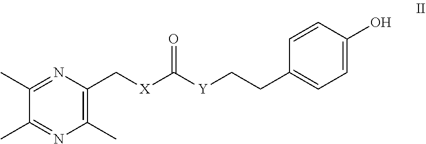

X and Y each are selected from the group consisting of O, S, Se, and NR₆.

In some embodiments, the pyrazine derivative may have the following structure:

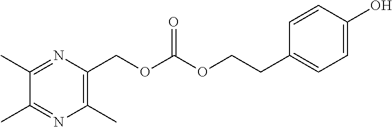 [SMILES: Cc1nc(C)c(COC(=O)OCCc2ccc(O)cc2)nc1C]

In some embodiments, the pyrazine derivative may have the following structure:

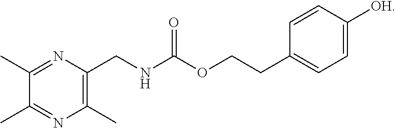 [SMILES: Cc1nc(C)c(CNC(=O)OCCc2ccc(O)cc2)nc1C]

In some embodiments, the pharmaceutically acceptable salt may be obtained by reaction of the compound with hydrochloric acid, sulfuric acid, phosphoric acid, hydrobromic acid, nitric acid, salicylic acid, oxalic acid, benzoic acid, maleic acid, fumaric acid, citric acid, succinic acid, tartaric acid, C₁₋₆ aliphatic carboxylic acid, C₁₋₆ alkyl sulfonic acid, benzenesulfonic acid, p-toluenesulfonic acid, or camphorsulfonic acid.

The present disclosure further provides a compound selected from the group consisting of the following compounds:

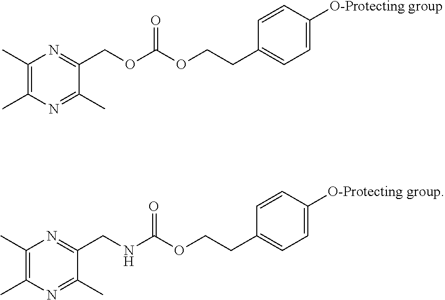

In some embodiments, the compound may be selected from the group consisting of the following compounds:

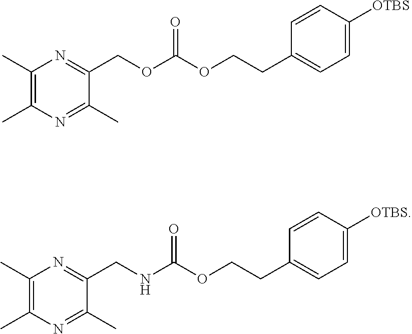 [SMILES: Cc1nc(C)c(COC(=O)OCCc2ccc(O[Si](C)(C)C(C)(C)C)cc2)nc1C ; Cc1nc(C)c(CNC(=O)OCCc2ccc(O[Si](C)(C)C(C)(C)C)cc2)nc1C]

The present disclosure further provides a method of preparation of a compound, including the following steps:

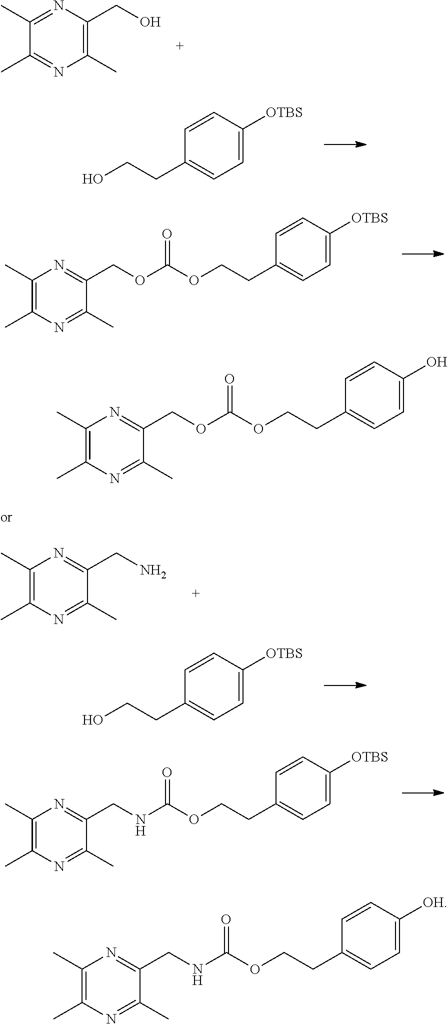

In some embodiments, the method of preparation may include the following steps:

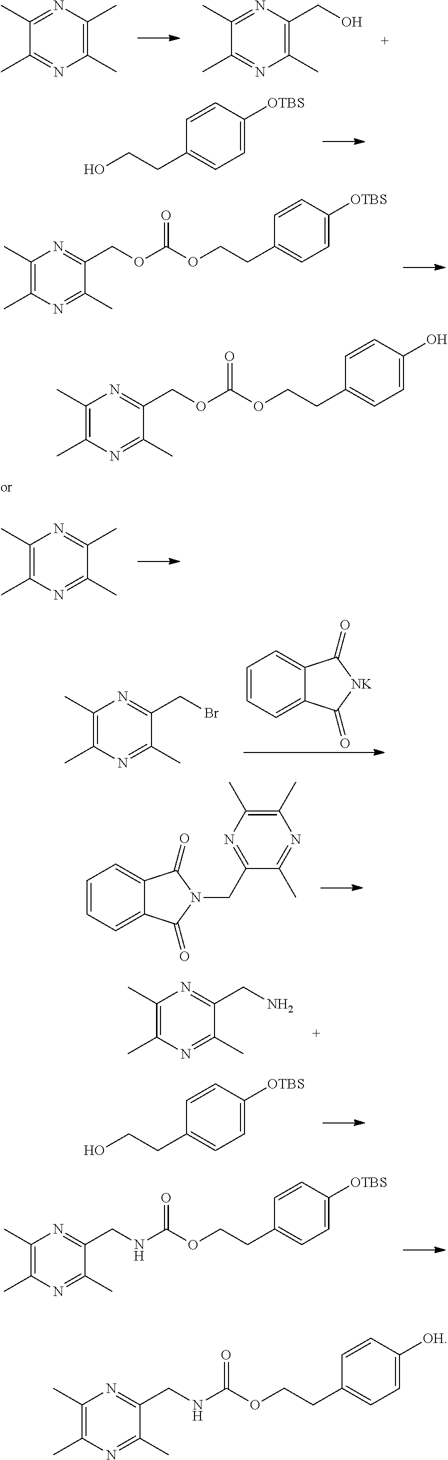

The present disclosure further provides a method of preparation of a compound, including the following steps:

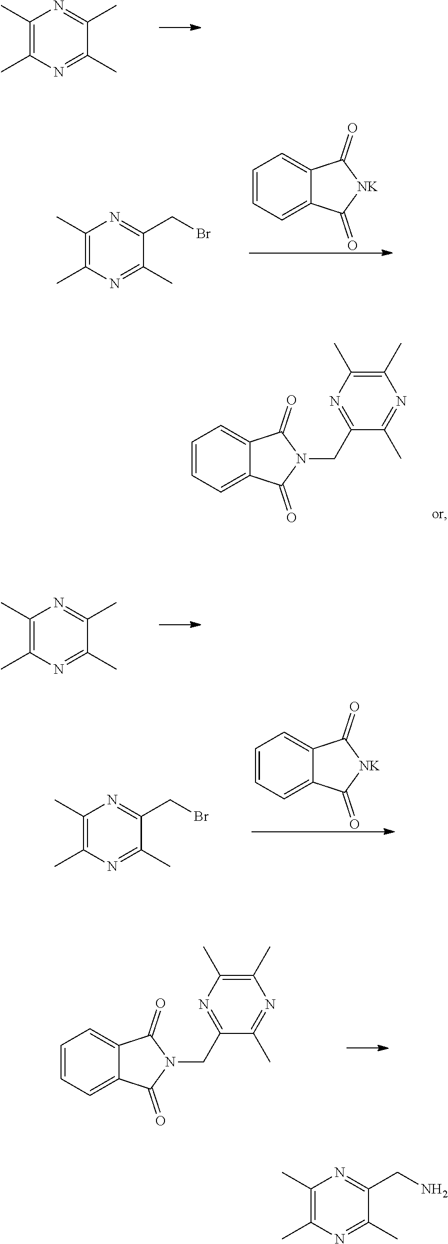

The present disclosure further provides a pharmaceutical composition, including a therapeutically effective amount of one or more of the pyrazine compound, the stereoisomer, the tautomer, and the pharmaceutically acceptable salt thereof.

The present disclosure further provides use of the pyrazine compound, the stereoisomer, the tautomer, and the pharmaceutically acceptable salt thereof in treating an ND selected from the group consisting of Alzheimer's disease, Parkinson's disease, Huntington's disease, FTD, vascular dementia, HIV-related dementia, multiple sclerosis, progressive lateral sclerosis, Friedreich's ataxia, neuropathic pain, or glaucoma, DM and a DM-related complication, an inflammation, an oxidative damage, and a mitochondrial disorder-related disease.

The pyrazine compound improves glucose and lipid metabolism, reduces urinary protein, and has a neuroprotective activity and anti-inflammatory properties. The pyrazine compound may also ameliorate memory impairment and anti-oxidative damage, have a therapeutic effect on amyotrophic lateral sclerosis (ALS), and prevent and/or treat Alzheimer's disease, Parkinson's disease and other diseases.

The present disclosure further provides a pharmaceutical composition, including a therapeutically effective amount of any one or more of the pyrazine compound, the stereoisomer, the tautomer, and the pharmaceutically acceptable salt thereof.

In some embodiments, the pharmaceutical composition may further include one or more pharmaceutically acceptable carriers or excipients.

In some embodiments, the pharmaceutical composition may further include other therapeutic agents.

In an embodiment of the present disclosure, the compound may be administered as a preparation of dosage unit containing a conventional pharmaceutically acceptable carrier by oral, injective, subcutaneous, respiratory, transdermal, parenteral, rectal, topical contact, intravenous, intramuscular administration, or other means. In some embodiments, the pharmaceutical composition may be prepared into a tablet, a granule, an injection, a gel, a pill, a capsule, a suppository, an implant, a nano preparation, and a powder for injection. Some dosage forms such as the tablet and the capsule may be subdivided into an appropriate unit dosage form containing an appropriate quantity of an active component, such as an effective amount to achieve a desired purpose.

The carrier includes excipients and diluents, and must have sufficiently high purity and sufficiently low toxicity to be suitable for administration to patients to be treated. The carrier may be inert or have a pharmaceutical benefit.

The carrier may include, but is not limited to, a diluent such as a filler, and a bulking agent, a binder, a lubricant, an anti-caking agent, a disintegrant, a sweetener, a buffer, a preservative, a solubilizer, an isotonic agent, a suspending agent and a dispersing agent, a wetting agent or an emulsifying agent, a flavoring agent and a perfuming agent, a thickening agent and a intermedium. In some embodiments, the pharmaceutically acceptable carrier may include sugar, starch, cellulose, malt, gelatin, talc, and vegetable oil. An optional activator may be included in the pharmaceutical composition, which do not substantially affect an activity of the compound in the present disclosure.

### Terminology:

A “stereoisomer” or “optical isomer” is a compound that has a same chemical composition but differs in arrangement of atoms or groups in space. The compound includes a “diastereomer” and an “enantiomer”.

The “diastereomer” is a stereoisomer that has two or more chiral centers, and molecules of the diastereomer are not mirror images of each other. The diastereomer has different physical properties such as melting point, boiling point, spectral properties and reactivity. A mixture of the diastereomers can be separated under high-resolution analytical steps such as electrophoresis and crystallization using a chiral HPLC column in the presence of a resolving agent or chromatography.

The “enantiomer” refers to two stereoisomers of a compound that are non-superimposable mirror images of each other. A 50:50 mixture of the enantiomers is called a racemic mixture or a racemate, which can occur during a chemical reaction or process where no stereoselectivity or stereospecificity is present.

The “alkyl” includes branched and straight-chain saturated aliphatic hydrocarbon groups and has the specified number of carbon atoms, typically 1 to about 12 carbon atoms. For example, the term C₁-C₆ alkyl as used herein refers to alkyl having 1 to about 6 carbon atoms. When C₀-Cₙ alkyl is used herein in conjunction with another group, (phenyl)C₀-C₄ alkyl is taken as an example to describe a designated group. In this case, the phenyl is bonded directly via a single covalent bond (C₀) or via an alkyl chain having the specified number of carbon atoms (in this case, 1 to about 4 carbon atoms). The alkyl includes, but is not limited to, methyl, ethyl, n-propyl, isopropyl, n-butyl, 3-methylbutyl, tert-butyl, n-pentyl, and sec-pentyl.

The “alkenyl” or “alkenyl” refers to straight and branched hydrocarbon chains including one or more unsaturated carbon-carbon bonds that may occur at any stable point along the chain. As used herein, the alkenyl generally has 2 to about 12 carbon atoms. In some embodiments, the alkenyl is lower alkenyl having 2 to about 8 carbon atoms, such as: C₂-C₈, C₂-C₆, and C₂-C₄ alkenyl. The alkenyl includes vinyl, propenyl, and butenyl.

The “cycloalkyl” refers to preferably alkyl with a monocyclic, bicyclic, tricyclic, bridged-cyclic and spirocyclic structure and having 3 to 15 carbon atoms, preferably including cyclopropane, cyclopentane, and cyclohexane.

The “alkoxy” refers to alkyl as defined above and having the specified number of carbon atoms attached through an oxygen bridge. The alkoxy includes, but is not limited to, methoxy, ethoxy, 3-hexyloxy, and 3-methylpentyloxy.

The “heterocycle” means a 5- to 8-membered saturated ring, a partially unsaturated ring, or an aromatic ring containing 1 to about 4 heteroatoms selected from N, O, and S, with C as the remaining ring atoms. The heterocycle can also be a 7- to 1l-membered saturated, partially unsaturated, or aromatic heterocyclic system, and a 10- to 15-membered tricyclic system; the system contains at least 1 heteroatom selected from N, O and S in a polycyclic system and up to about 4 heteroatoms independently selected from N, O and S in each ring of the polycyclic system. Unless otherwise specified, a heterocycle can be attached to a group, in which any heteroatom and carbon atom on the heterocycle is substituted with the group and thereby results in a stable structure. When specified, the heterocycle herein may be substituted on carbon atom or nitrogen atom so long as the resulting compound is stable. Optionally, nitrogen atoms in the heterocycle can be quaternized. Preferably, not more than 4 heteroatoms are in heterocyclyl; preferably, not more than 2, more preferably not more than 1 of S and O atoms are in heterocyclyl. Examples of the heterocyclyl includes pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, benz[b]thiophenyl, isoquinolinyl, quinazolinyl, quinoxalinyl, thienyl, isoindolyl, dihydroisoindolyl, 5,6,7,8-tetrahydroisoquinoline, pyridyl, pyrimidinyl, furanyl, thienyl, pyrrolyl, pyrazolyl, pyrrolidinyl, morpholinyl, piperazinyl, piperidinyl, and pyrrolidinyl.

The “aryl” or “heteroaryl” means a stable 5- or 6-membered monocyclic ring or polycyclic ring containing 1 to 4, or preferably 1 to 3 heteroatoms selected from N, O and S with C as the remaining ring atoms. When the total number of S and O atoms in heteroaryl exceeds 1, these heteroatoms are not adjacent to each other. Preferably, the total number of S and O atoms in heteroaryl is not greater than 2. Especially preferably, the total number of S and O atoms in heteroaryl is not greater than 1. Optionally, nitrogen atoms in the heterocycle can be quaternized. When specified, these heteroaryl may also be substituted with carbon or non-carbon atoms or groups. Such substitution may include fusing with a 5- to 7-membered saturated ring group optionally containing 1 or 2 heteroatoms independently selected from N, O, and S to form, for example, [1,3]dioxin azolo[4,5-c]pyridyl. The heteroaryl includes, but is not limited to, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolyl, pyrrolyl, pyrazolyl, benzo[b]phenylthio, isoquinolinyl, quinazolinyl, quinoxalinyl, thienyl, isoindolyl, and 5,6,7,8-tetrahydroisoquinoline.

The “pharmaceutically acceptable salt” or “salt of compound” are derivatives of the disclosed compounds, where the parent compound is modified by preparing a non-toxic acid or base addition salts thereof; the two terms also refer to a pharmaceutically acceptable solvate, including hydrates, of these compounds and these salts. The pharmaceutically acceptable salt includes, but is not limited to, inorganic or organic acid addition salts of basic residues such as amines, base or organic addition salts of acidic residues such as carboxylic acid, and combinations of one or more of the above salts. The pharmaceutically acceptable salt includes nontoxic and quaternary ammonium salts such as a parent compound formed from non-toxic inorganic or organic acids. For example, the non-toxic acid salt includes those derived from inorganic acids, such as hydrochloric acid, hydrobromic acid, sulfuric acid, sulfamic acid, phosphoric acid, nitric acid; other acceptable inorganic salt includes metal salts, such as sodium salts, potassium salts, cesium salts; alkaline earth metal salt includes: calcium salts and magnesium salts, and combinations of one or more of the above salts.

An organic salt of the compounds includes those prepared from organic acids such as acetic acid, trifluoroacetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, methanesulfonic acid, ethanesulfonic acid, benzenesulfonic acid, p-aminobenzenesulfonic acid, 2-acetoxybenzoic acid, fumaric acid, p-toluenesulfonic acid, methanesulfonic acid, ethanedisulfonic acid, oxalic acid, isethionic acid, HOOC—(CH₂)n-COOH (where n is 0 to 4); organic amine salts, such as: triethylamine salts, pyridine salts, picoline salts, ethanolamine salts, triethanolamine salts, dicyclohexylamine salts, and N,N′-dibenzylethylenediamine salts; and amino acid salts, such as arginine, aspartate, and glutamate, and combinations of one or more of the above salts.

### BRIEF DESCRIPTION OF THE DRAWINGS

FIG. 1 shows that OLB-1 and OLB-2 significantly reduce death of SH—SY5Y cells caused by oxygen glucose deprivation (OGD);

FIG. 2 shows that OLB-1 and OLB-2 significantly reduce a urinary protein level of db/db mice;

FIG. 3 shows that OLB-1 and OLB-2 significantly reduce a level of a pro-inflammatory factor in hippocampus of 5*FAD mice;

FIG. 4 shows that OLB-1 and OLB-2 significantly reduce a level of a pro-inflammatory factor in hippocampus of 5*FAD mice;

FIG. 5 shows that OLB-1 and OLB-2 significantly improve memory impairment in the 5*FAD mice;

FIG. 6 shows effects of OLB-1 and OLB-2 on pole climbing time of ALS transgenic mice;

FIG. 7 shows effects of OLB-1 and OLB-2 on a limb grip force of the ALS transgenic mice; and

FIG. 8 shows that OLB-1 and OLB-2 significantly reduce the number of rotations in APO-induced 6-OHDA Parkinson's disease rats.

### DETAILED DESCRIPTION OF THE EMBODIMENTS

### Example 1 Synthesis of Compound OLB-1

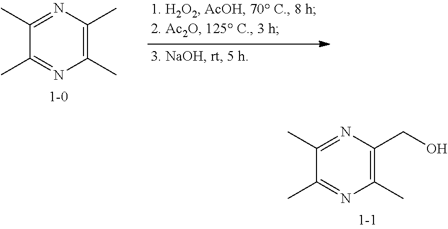

Step (1): a compound 1-0 (15.0 g, 110.3 mmol) was dissolved in glacial acetic acid (150 ml), hydrogen peroxide (30%, 12.5 ml, 110.2 mmol) was added dropwise at 70° C., and the reaction was continued overnight. After the reaction, a resulting product was cooled, diluted with an aqueous sodium hydroxide solution (50%), extracted with dichloromethane, dried over anhydrous sodium sulfate, filtered, and concentrated to obtain a crude compound, and the crude compound was directly dissolved in acetic anhydride (30 ml), and reacted at 107° C. for 3 h; an obtained product was cooled, concentrated, diluted with ice water, adjusted to a pH value of greater than 10 with a sodium hydroxide solution, stirred overnight, extracted with dichloromethane, dried over anhydrous sodium sulfate, filtered, concentrated, and subjected to silica gel column chromatography to obtain a product 1-1 (6.8 g, 41%). ¹H NMR (400 MHz, DMSO-d₆) δ 5.39 (s, 1H), 3.81 (s, 2H), 2.42 (s, 3H), 2.42 (s, 3H), 2.41 (s, 3H). MS (ESI) m/z: 153.1 [M+H]⁺.

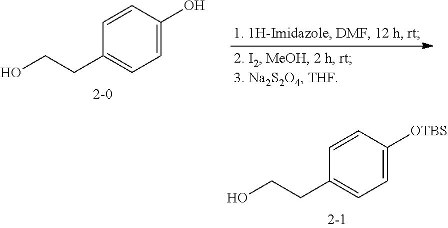 [SMILES: OCCc1ccc(O)cc1 ; CC(C)(C)[Si](C)(C)Oc1ccc(CCO)cc1]

Step (2): Compounds imidazole (6.2 g, 90.5 mmol) and tert-butyldimethylsilyl chloride (13.6 g, 90.5 mmol) were dissolved in N,N-dimethylformamide (200 ml) a compound 2-0 (5.0 g, 36.2 mmol) was added in portions, and stirred at room temperature for reaction overnight. After the reaction, a resulting product was diluted with water, extracted with n-hexane, dried over anhydrous sodium sulfate, filtered and concentrated; a part (3.7 g) of an obtained crude product was dissolved in methanol (40 ml), and elemental iodine (0.4 g) was added and stirred for 2 h; after the reaction, a resulting product was quenched with sodium thiosulfate, concentrated, diluted with ether, washed with water and then saturated brine, dried over anhydrous sodium sulfate, filtered, concentrated, and subjected to silica gel column chromatography to obtain a product 2-1 (2.0 g, 83%). ¹H NMR (400 MHz, DMSO-d₆) δ 6.92 (d, J=8.5 Hz, 2H), 6.69-6.49 (m, 2H), 4.41 (t, J=5.2 Hz, OH), 3.40 (td, J=7.1, 5.3 Hz, 2H), 2.49 (t, J=7.1 Hz, 2H), 0.78 (s, 9H), 0.07 (s, 6H). MS (ESI) m/z: 253.2 [M+H]⁺.

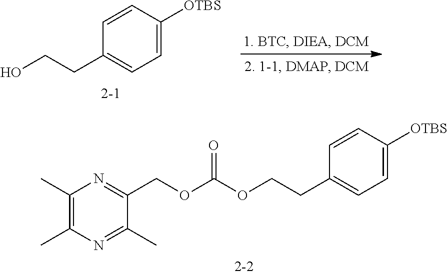 [SMILES: CC(C)(C)[Si](C)(C)Oc1ccc(CCO)cc1 ; Cc1nc(C)c(COC(=O)OCCc2ccc(O[Si](C)(C)C(C)(C)C)cc2)nc1C]

Step (3): Under nitrogen protection, the compound 2-1 (252 mg, 1 mmol) and triphosgene (112 mg, 0.34 mmol) were dissolved in anhydrous dichloromethane (15 ml), N,N-diisopropylethylamine (0.1 ml) was added, stirred at room temperature for 0.5 h, and the compound 1-1 (304 mg, 2 mmol) and 4-dimethylaminopyridine (366 mg, 3 mmol) were added in sequence, and the reaction was continued overnight at room temperature. A resulting product was concentrated and subjected to silica gel column chromatography to obtain a product 2-2 (241 mg, 56%). ¹H NMR (400 MHz, CDCl₃) δ 6.88 (d, J=8.4 Hz, 2H), 6.58 (d, J=8.4 Hz, 2H), 5.05 (s, 2H), 4.14 (t, J=7.2 Hz, 2H), 2.73 (t, J=7.2 Hz, 2H), 2.35 (s, 1H), 2.31 (s, 1H), 2.31 (s, 1H), 0.79 (s, 9H), 0.00 (s, 6H). MS (ESI) m/z: 431.2 [M+H]V.

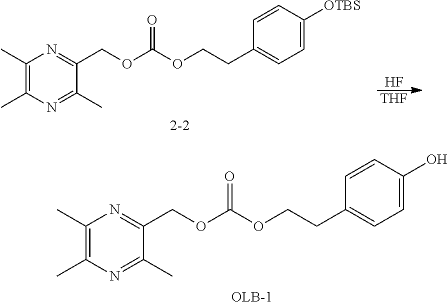

Step (4): the compound 2-2 (86 mg, 0.2 mmol) was dissolved in tetrahydrofuran (10 ml), hydrofluoric acid solution (1.0 ml, 2.0 mmol) was added, and a resulting mixture was refluxed for 1 h. After the reaction, a resulting product was washed with a saturated sodium bicarbonate solution, water and saturated brine in sequence, the organic phase was dried with anhydrous sodium sulfate, filtered, concentrated, and a product OLB-1 (54 mg, 86%) was obtained by silica gel column chromatography. ¹H NMR (400 MHz, CDCl₃) δ 7.02 (d, J=8.5 Hz, 2H), 6.74 (d, J=8.5 Hz, 2H), 5.24 (s, 2H), 4.30 (t, J=7.2 Hz, 2H), 2.88 (t, J=7.2 Hz, 2H), 2.53 (s, 1H), 2.51 (s, 1H), 2.50 (s, 1H). MS (ESI) m/z: 317.2 [M+H]⁺.

### Example 2 Synthesis of Compound OLB-2

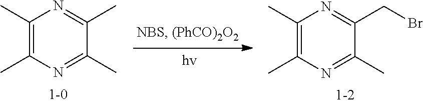

Step (1): a compound 1-0 (20 g, 147 mmol), N-bromosuccinimide (26.7 g, 150 mmol) and benzoyl peroxide (50 mg, 0.2 mmol) were dissolved in carbon tetrachloride (70 ml); under the irradiation of an incandescent lamp, a resulting mixture was refluxed for 10 h; and a product was filtered and concentrated to obtain a crude product 1-2, which directly participated in a next reaction. ¹H NMR (400 MHz, CDCl₃) δ 4.54 (s, 2H), 2.57 (s, 1H), 2.50 (s, 1H), 2.49 (s, 1H).

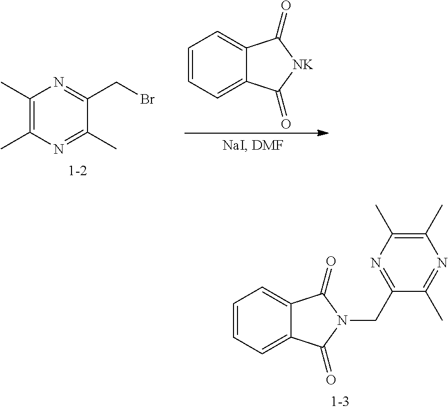

Step (2): the compound 1-2 (17.5 g, 82 mmol), potassium phthalimide (21.0 g, 110 mmol) and sodium iodide (0.5 g, 3.3 mmol) were dissolved in N,N-dimethylformamide (100 ml), stirred at 95° C. for 2 h. After the reaction, a resulting product was filtered, and a filtrate was poured into ice water to obtain a white precipitate, which was filtered by suction, and a filter cake was recrystallized from ethanol to obtain a product 1-3 (18.7 g, 81%). ¹H NMR (400 MHz, DMSO-d₆) δ 8.01-7.79 (m, 4H), 4.90 (s, 2H), 2.53 (s, 3H), 2.38 (s, 3H), 2.21 (s, 3H). MS (ESI) m/z: 282.1 [M+H]⁺.

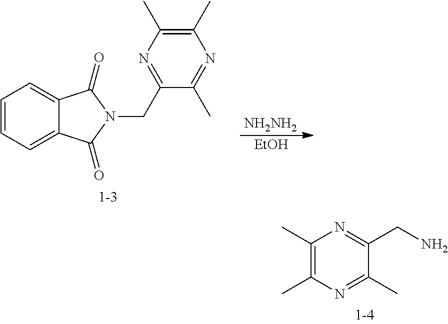

Step (3): the compound 1-3 (2.8 g, 10 mmol) was dissolved in ethanol (30 ml), hydrazine hydrate (50%, 1.0 ml) was added, and refluxed for 2 h. After the reaction, a resulting product was filtered, adjusted to a pH value of 1 to 2 with hydrochloric acid, filtered, concentrated, stirred with a sodium hydroxide solution (20%), extracted with dichloromethane, and concentrated to obtain a product 1-4 (0.83 g, 55%). ¹H NMR (400 MHz, DMSO-d₆) δ 8.07 (dd, J=5.9, 3.3 Hz, 1H), 7.84 (dd, J=5.9, 3.3 Hz, 1H), 3.81 (s, 2H), 2.42 (s, 3H), 2.42 (s, 3H), 2.41 (s, 3H). MS (ESI) m/z: 152.1 [M+H]⁺.

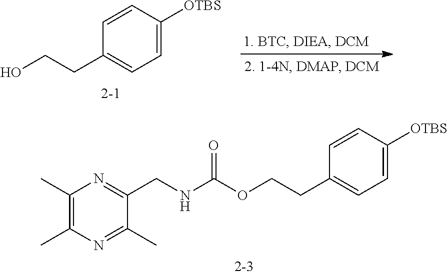 [SMILES: CC(C)(C)[Si](C)(C)Oc1ccc(CCO)cc1 ; Cc1nc(C)c(CNC(=O)OCCc2ccc(O[Si](C)(C)C(C)(C)C)cc2)nc1C]

Step (4): Under nitrogen protection, the compound 2-1 (252 mg, 1 mmol) and triphosgene (112 mg, 0.34 mmol) were dissolved in anhydrous dichloromethane (15 ml), N,N-diisopropylethylamine (0.1 ml) was added, stirred at room temperature for 0.5 h, and the compound 1-4 (302 mg, 2 mmol) and 4-dimethylaminopyridine (366 mg, 3 mmol) were added in sequence, and the reaction was continued overnight at room temperature. A resulting product was concentrated and subjected to silica gel column chromatography to obtain a product 2-3 (265 mg, 62%). ¹H NMR (400 MHz, CDCl₃) δ 6.90 (d, J=8.4 Hz, 2H), 6.58 (d, J=8.4 Hz, 2H), 4.24 (d, J=3.5 Hz, 2H), 4.11 (t, J=7.1 Hz, 2H), 2.71 (t, J=7.1 Hz, 2H), 2.30 (s, 9H), 0.79 (s, 9H), 0.00 (s, 6H). MS (ESI) m/z: 430.2 [M+H]⁺.

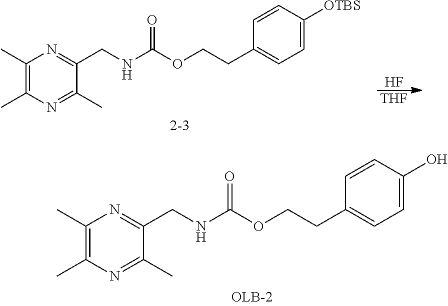

Step (5): the compound 2-3 (85 mg, 0.2 mmol) was dissolved in tetrahydrofuran (10 ml), hydrofluoric acid solution (1.0 ml, 2.0 mmol) was added, and a resulting mixture was refluxed for 1 h. After the reaction, a resulting product was washed with a saturated sodium bicarbonate solution, water and saturated brine in sequence, the organic phase was dried with anhydrous sodium sulfate, filtered, concentrated, and a product OLB-2 (51 mg, 81%) was obtained by silica gel column chromatography. ¹H NMR (400 MHz, CDCl₃) δ 7.06 (d, J=8.3 Hz, 2H), 6.76 (d, J=8.3 Hz, 2H), 4.42 (d, J=3.8 Hz, 2H), 4.28 (t, J=7.0 Hz, 2H), 2.88 (t, J=7.1 Hz, 2H), 2.42 (s, 3H), 2.42 (s, 3H), 2.41 (s, 3H). MS (ESI) m/z: 316.2 [M+H]⁺.

### Example 3 OLB-1 and OLB-2 Significantly Reducing SH—SY5Y Cell Death Caused by OGD

Neuroprotective effect of tetramethylpyrazine (TMP) and derivatives thereof was evaluated by MTT assay. Cells were incubated, log-phase cells were collected, a concentration of a cell suspension was adjusted, an MTT-containing medium was added after chemical treatment and 4 h of incubation with OGD, incubation was conducted for 4 h, the medium in the wells was carefully removed, and 150 μl of dimethyl sulfoxide (DMSO) was added to each well, shaken at a low speed for 10 min on a shaker to fully dissolve crystals, and an absorbance value of each well was measured at an OD (absorbance) value of 490 nm of an enzyme-linked immunosorbent assay instrument (at the same time, a zero adjustment well (medium, MTT, DMSO), a control well (cells, a drug dissolution medium with the same concentration, medium, MTT, DMSO)). Data were presented as mean±SEM; n=8 per group. One-way ANOVA and multiple range test were adopted to show differences between the two groups. a, p<0.001 vs. control group; b, p<0.05 vs. OGD group; c, p<0.001 vs. OGD group.

It can be seen from FIG. 1 that OLB-1 and OLB-2 could significantly reduce the death of SH—SY5Y cells caused by OGD and have neuroprotective effects.

### Example 4 OLB-1 and OLB-2 Significantly Reducing Elevation of Inflammatory Factors and Oxidative Stress Caused by LPS

The SH—SY5Y cells were recovered and cultured, and the cells in the logarithmic growth phase were taken. After 24 h of incubation, SH—SY5Y neuroblastoma cells were treated with 1 μM all-trans retinoic acid to induce differentiation, and then inoculated into a 6-well culture dish for 24 h of incubation. The medium was treated with chemicals feed at 0.2 μM (L) or 1 μM (H) and 1 μg/mL LPS for 24 h, a supernatant medium was aspirated, and changes of inflammatory factors and oxidative stress-related proteins were measured by an ELISA kit. Data were presented as mean±SEM; n=8 per group. One-way ANOVA and multiple range test were adopted to show differences between the two groups. a, p<0.05 vs. LPS group; b, p<0.01 vs. LPS group; c, p<0.001 vs. LPS group.

TABLE 1

| (pg/ml) | WT | LPS | LPS + TMP(L) | LPS + TMP(H) |
|---|---|---|---|---|
| TNF-alpha | 0.00 | 12.85 ± 1.45 | 12.22 ± 1.28 | 12.64 ± 1.70 |
| IL1 | 0.00 | 13.59 ± 1.63 | 13.26 ± 1.31 | 13.53 ± 1.74 |
| IL6 | 0.00 | 13.92 ± 1.13 | 14.12 ± 1.46 | 12.69 ± 1.85 |
| (pg/ml) | LPS + OLB01(L) | LPS + OLB01(H) | LPS + OLB02(L) | LPS + OLB02(H) |
| TNF-alpha | 9.64 ± 1.56(a) | 6.78 ± 1.67(c) | 9.92 ± 1.44(b) | 6.99 ± 1.31(c) |
| IL1 | 10.94 ± 1.48(a) | 8.46 ± 1.33(b) | 10.34 ± 1.89(a) | 7.37 ± 1.58(c) |
| IL6 | 11.60 ± 1.39(a) | 7.97 ± 1.61(c) | 11.05 ± 1.87(a) | 8.19 ± 1.24(c) |

TABLE 2

| | WT | LPS | LPS + TMP(L) | LPS + TMP(H) |
|---|---|---|---|---|
| SOD(nU/ml) | 20.49 ± 1.09 | 9.40 ± 0.77 | 9.57 ± 0.61 | 8.93 ± 0.47 |
| MDA(nmol/mg) | 0.35 ± 0.02 | 23.75 ± 1.29 | 23.34 ± 1.71 | 22.94 ± 1.33 |
| GSH-Px(umol/mg) | 20.59 ± 1.05 | 5.90 ± 0.58 | 5.96 ± 0.74 | 6.90 ± 0.87 |
| | LPS + OLB01(L) | LPS + OLB01(H) | LPS + OLB02(L) | LPS + OLB02(H) |
| SOD(nU/ml) | 12.58 ± 0.72(b) | 16.76 ± 0.38(c) | 12.13 ± 0.65(b) | 15.16 ± 1.52(c) |
| MDA(nmol/mg) | 16.15 ± 1.26(c) | 10.51 ± 0.74(c) | 15.75 ± 0.95(c) | 9.37 ± 0.73(c) |
| GSH-Px(umol/mg) | 8.39 ± 0.69(c) | 13.71 ± 0.84(c) | 7.93 ± 1.05(b) | 13.40 ± 1.24(c) |

From Table 1 and Table 2, it can be seen that OLB-1 and OLB-2 significantly reduced the elevation of inflammatory factors and oxidative stress caused by LPS, and had strong anti-inflammatory and antioxidant effects.

### Example 5 OLB-1 and OLB-2 Significantly Improving Abnormal Glucose and Lipid Metabolism in db/db Mice

Mice of a normal control group and model mice were given normal saline 10 ml/kg/d, metformin hydrochloride enteric-coated tablets 225 mg/kg/d, Losartan 10 mg/kg, TMP (5.0 mg/kg, 0.037 mmol/kg), and OLB-1 and OLB-2 (13.32 mg/kg, 0.037 mmol/kg), at a volume of 10 mL/kg, once/d, for continuous administration of 56 d, blood lipids and blood glucose related indexes were measured after blood collection. Data were presented as mean±SEM; n=6 per group. One-way ANOVA and multiple range test were adopted to show differences between the two groups. a, p<0.05 vs. db/db group; c, p<0.001 vs. db/db group.

TABLE 3

| | Glycated | Glucose | Total | Triglyceride |
|---|---|---|---|---|
| | hemoglobin (nmol/L) | determination (mM) | cholesterol (mM) | (mg/dL) |
| WT | 184.47 ± 10.16 | 7.75 ± 1.55 | 2.42 ± 0.11 | 9.11 ± 1.28 |
| db/db | 983.37 ± 41.26 | 39.71 ± 3.03 | 12.4 ± 1.05 | 13.81 ± 1.19 |
| db/db + metformin | 628.52 ± 50.59(c) | 24.01 ± 2.38(c) | / | / |
| db/db + Losartan | / | / | 6.76 ± 0.46(c) | 9.68 ± 0.37(c) |
| db/db + TMP | 917.31 ± 57.43 | 37.94 ± 3.11 | 10.96 ± 1.38 | 12.83 ± 1.37 |
| db/db + OLB1 | 643.06 ± 61.53(c) | 25.99 ± 4.46(c) | 8.36 ± 2.63(c) | 10.88 ± 0.92(b) |
| db/db + OLB2 | 595.38 ± 52.47(c) | 26.61 ± 1.15(c) | 7.38 ± 0.34(c) | 10.65 ± 0.54(b) |
| | High-density lipoprotein | Low-density lipoprotein | Urea | Creatinine |
| | cholesterol (mM) | cholesterol (mM) | (mmol/L) | (μmol/L) |
| WT | 1.45 ± 0.13 | 0.44 ± 0.03 | 6.86 ± 0.25 | 38.25 ± 1.33 |
| db/db | 2.365 ± 0.16 | 0.71 ± 0.03 | 12.06 ± 0.81 | 54.62 ± 3.34 |
| db/db + metformin | 2.19 ± 0.12(a) | 0.59 ± 0.02(c) | / | / |
| db/db + Losartan | / | / | 7.23 ± 0.51(c) | 42.12 ± 1.72(c) |
| db/db + TMP | 2.49 ± 0.16 | 0.68 ± 0.04 | 11.55 ± 0.59 | 52.5 ± 3.76 |
| db/db + OLB1 | 1.81 ± 0.15(b) | 0.69 ± 0.04(c) | 8.03 ± 0.61(c) | 48.7 ± 3.68(a) |
| db/db + OLB2 | 1.87 ± 0.18(b) | 0.51 ± 0.03(c) | 7.48 ± 0.35(c) | 46.28 ± 2.76(b) |

As can be seen from Table 3, OLB-1 and OLB-2 significantly ameliorated abnormal glucose and lipid metabolism, decreased total cholesterol and triglyceride, decreased high-density lipoprotein cholesterol and low-density lipoprotein cholesterol, and decreased urea and creatinine.

### Example 6 OLB-1 and OLB-2 Significantly Reducing Urinary Protein Levels in db/db Mice

Mice of a normal control group and model mice were given normal saline 10 ml/kg/d, Losartan 10 mg/kg, TMP (5.0 mg/kg, 0.037 mmol/kg), OLB-1 (11.7 mg/kg, 0.037 mmol/kg), and OLB-2 (11.67 mg/kg, 0.007 mmol/kg), at a volume of 10 mL/kg, once/d, for continuous administration of 90 d, urine protein levels were measured after urine collection. Data were presented as mean±SEM; n=6 per group. One-way ANOVA and multiple range test were adopted to show differences between the two groups. a, p<0.05 vs. db/db group; c, p<0.001 vs. db/db group.

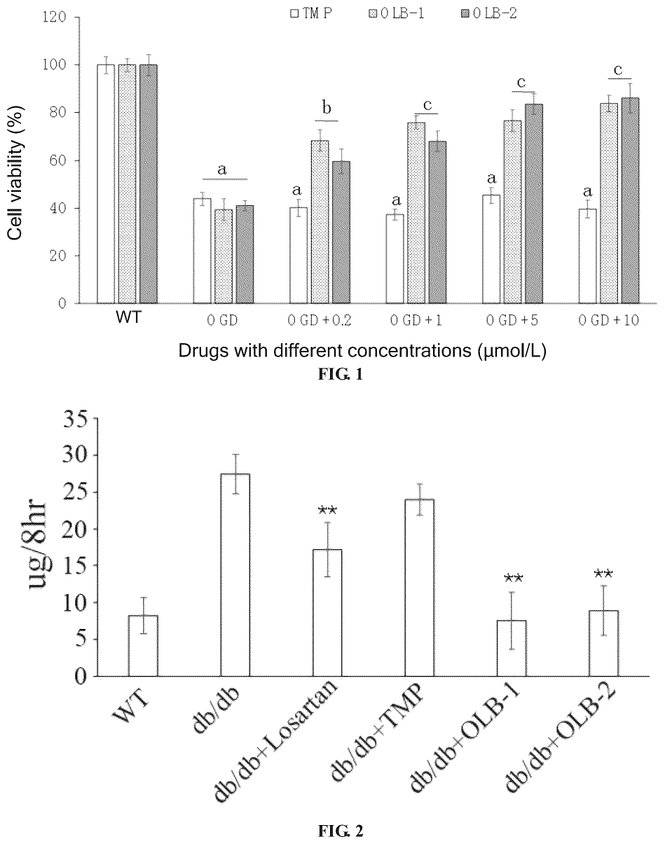

As shown in FIG. 2, OLB-1 and OLB-2 significantly reduced urinary protein levels.

### Example 7 OLB-1 and OLB-2 Significantly Improving Biochemical and Metabolic Indicators in db/db Mice

Mice of a normal control group and model mice were given normal saline 10 ml/kg/d, Losartan 15 mg/kg/d, TMP (5.0 mg/kg, 0.037 mmol/kg), OLB-1 (2.31 mg/kg, 0.007 mmol/kg), OLB-2 (2.3 mg/kg, 0.007 mmol/kg), at a volume of 10 mL/kg, once/d, for continuous administration of 90 d, blood lipids and blood sugar related indicators were measured. Data were presented as mean±SEM; n=6 per group. One-way ANOVA and multiple range test were adopted to show differences between the two groups. a, p<0.05 vs. db/db group; b, p<0.01 vs. db/db group; c, p<0.001 vs. db/db group.

TABLE 4

| | Urine albumin/ | Urea | Creatinine |
|---|---|---|---|
| | creatinine (mg/g) | (mmol/L) | (μmol/L) |
| WT | 46.57 ± 12.31 | 6.86 ± 0.25 | 38.25 ± 1.33 |
| db/db | 771.16 ± 98.73 | 12.06 ± 0.81 | 54.62 ± 3.34 |
| db/db + | 522.59 ± 103.42(c) | 7.23 ± 0.51(c) | 42.12 ± 1.72(c) |
| Losartan | | | |
| db/db + | 687.35 ± 79.04 | 11.55 ± 0.59 | 52.5 ± 3.76 |
| TMP | | | |
| db/db + | 438.69 ± 83.23(c) | 8.03 ± 0.61(c) | 48.7 ± 3.68(a) |
| OLB1 | | | |
| db/db + | 477.51 ± 90.68(c) | 7.48 ± 0.35(c) | 46.28 ± 2.76(b) |
| OLB2 | | | |

As can be seen from the above table, OLB-1 and OLB-2 significantly decreased the biochemical and metabolic indicators of db/db mice, and decreased urea and creatinine.

### Example 8 Use of OLB-1 and OLB-2 in ND

### OLB-1 and OLB-2 Significantly Reducing Level of Pro-Inflammatory Factor in Hippocampus of 5*FAD Mice

After 6-month-old 5*FAD mice were treated with OLB-1 and OLB-2 for 3 months, the levels of IL-1β (A) and TNFα (B) in mouse hippocampus were detected by ELISA. 5*FAD mice were treated with low-dosage and high-dosage OLB-1 (low dosage: 2.31 mg/kg, 0.007 mmol/kg; high dosage: 11.70 mg/kg, 0.037 mmol/kg, the same below) and OLB-2 (low dosage: 2.3 mg/kg, 0.007 mmol/kg; high dosage: 11.67 mg/kg, 0.037 mmol/kg, the same below) and TMP (5.0 mg/kg, 0.037 mmol/kg, the same below). Data were presented as mean±SEM; n=5 to 6 per group. One-way ANOVA and multiple range test were adopted to show differences between the two groups. **p<0.01, ***p<0.001 vs. WT group; #p<0.05, ##p<0.01, ###p<0.001 vs. 5*FAD group.

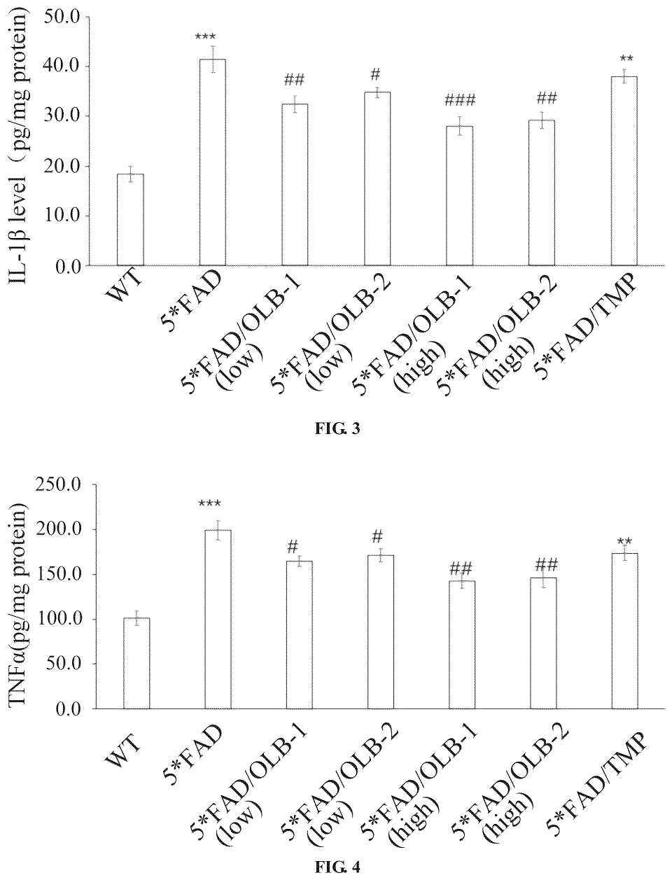

As shown in FIG. 3 and FIG. 4, OLB-1 and OLB-2 significantly reduced the proinflammatory cytokines TNF-α and IL-1β.

### OLB-1 and OLB-2 Significantly Improving Memory Impairment in 5*FAD Mice

After 6-month-old 5*FAD mice were treated with OLB-1 and OLB-2 for 3 months, the number of errors that the mice jumped off the platform was detected by electrical jumping. 5*FAD mice were treated with low-dosage and high-dosage OLB-1 (low dosage: 2.31 mg/kg, 0.007 mmol/kg; high dosage: 11.70 mg/kg, 0.037 mmol/kg) and OLB-2 (low dosage: 2.3 mg/kg, 0.007 mmol/kg; high dosage: 11.67 mg/kg, 0.037 mmol/kg) and TMP (5.0 mg/kg, 0.037 mmol/kg, the same below). Data were presented as mean±SEM; n=10 per group. One-way ANOVA and multiple range test were adopted to show differences between the two groups. ***p<0.001 vs. WT group; #p<0.05, ##p<0.01 vs. 5*FAD group.

As shown in FIG. 5, OLB-1 and OLB-2 significantly improve memory impairment.

### Effects of OLB-1 and OLB-2 on Pole Climbing Time of ALS Transgenic Mice

The pole climbing test is generally used to evaluate a motor coordination ability and motor delay of the limbs in mice. A homemade wooden pole about 50 cm long and about 1 cm in diameter was wrapped with medical gauze to increase friction of the wooden pole. The wooden pole was put vertically on a horizontal table, the mouse tail was grabbed such that the mouse head was facing down, with limbs grabbing a top of the pole; after releasing the mouse tail, timing was started, and it was ensured that the mouse crawled downward without external force, and the time was recorded for the mouse to climb from the top of the pole to a bottom platform (a standard was that mouse hind limbs were landed on the ground). Mice were continuously trained on this behavior for 3 d before administration, each mouse was repeated three times, and the mice that did not meet the standard were excluded. After the start of administration, the mouse behavior was tested every two weeks, a maximum of test results did not exceed 15 sec, and values exceeding 15 sec were recorded as 15 sec. An average of the three pole climbing times of mice was calculated as a final pole climbing time. ALS (SOD-G93A) transgenic mice develop obvious bradykinesia after the onset of disease, manifested as pole climbing time was significantly longer than that of control mice, and the bradykinesia became more severe with age. After treatment with different dosages of OLB-1, OLB-2, TMP and riluzole, it was found that OLB-1, OLB-2 and the positive control drug riluzole (5 mg/kg) each could significantly improve the symptoms of bradykinesia. Data were presented as mean±SEM; n=10 per group. One-way ANOVA and multiple range test were adopted to show differences between the two groups. ***p<0.001 vs. WT (normal control) group; #p<0.05, ##p<0.01 vs. ALS (SOD-G93A) group.

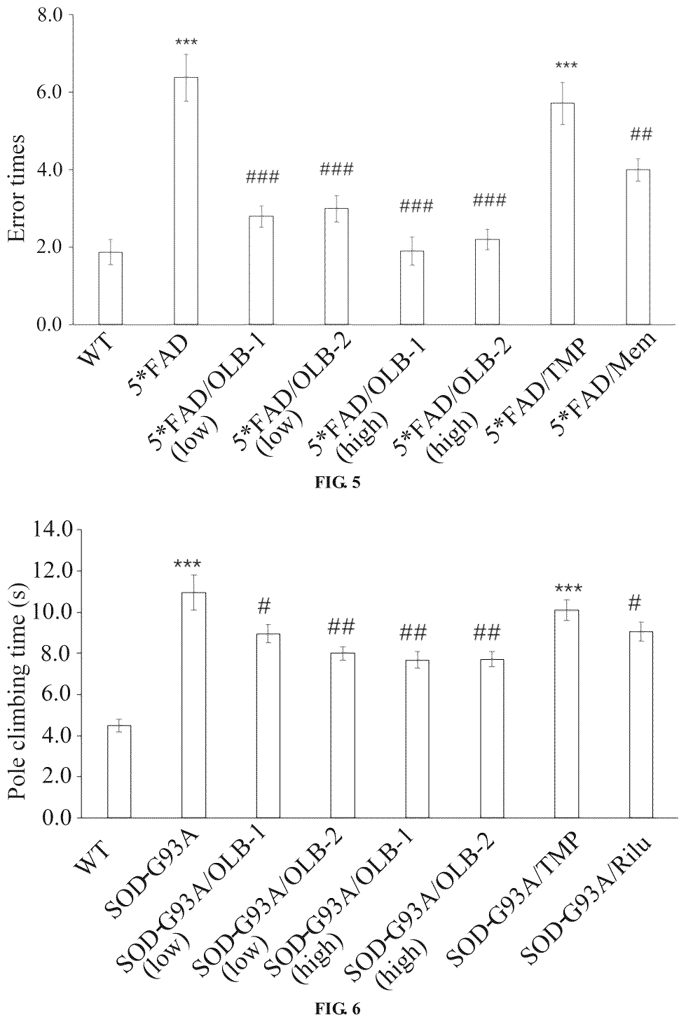

As shown in FIG. 6, OLB-1 and OLB-2 had therapeutic effects on ALS, significantly shortening pole climbing time and improving bradykinesia.

### Effects of OLB-1 and OLB-2 on Limb Grip Force of ALS Transgenic Mice

The limb grip force test is used directly to assess muscle strength in mice. The mouse were placed on a central stage of a grip board, the mouse tail was gently pulled to urge the mouse to grasp the grip board, and the mouse was pulled backward and horizontally when the mouse firmly grasped a grip net, and the data were recorded when the instrument showed a maximum grip force. After the start of administration, the grip force of the mice was tested every two weeks, and the measurement was repeated three times for each mouse, and the maximum among the three results was taken as a maximum grip force of the mice. After the ALS transgenic mice enter the disease stage, the limb grip force was significantly smaller than that of the WT mice. After treatment with different dosages of OLB-1, OLB-2, TMP and riluzole, it was found that OLB-1, OLB-2 and the positive control drug riluzole (5 mg/kg) each could effectively increase the limb force of mice, and delays the deterioration of limb grip force decline in ALS mice. Data were presented as mean±SEM; n=10 per group. One-way ANOVA and multiple range test show differences between the two groups. **p<0.01, ***p<0.001 vs. WT (normal control) group; #p<0.05, ##p<0.01 vs. ALS (SOD-G93A) group.

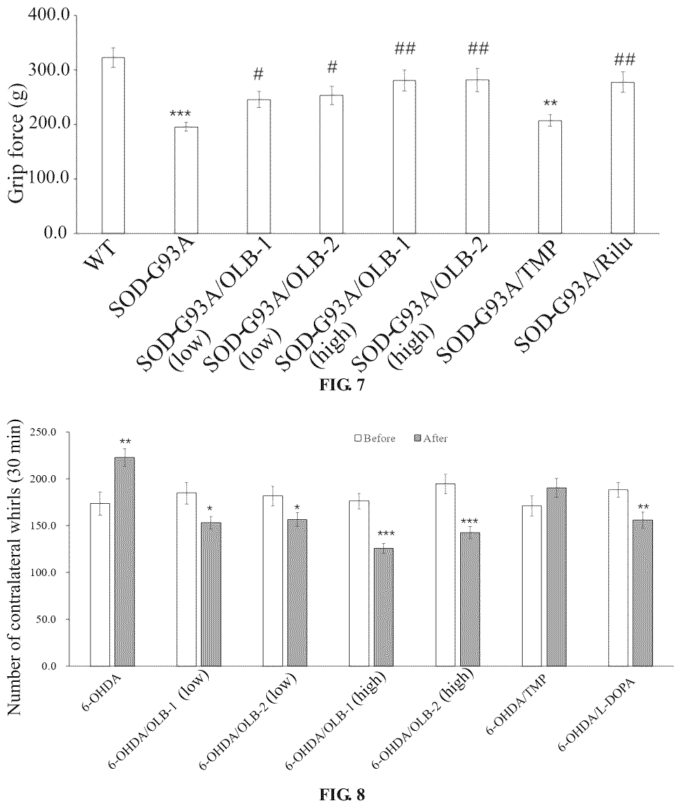

As shown in FIG. 7, OLB-1 and OLB-2 had therapeutic effects on ALS, significantly improving limb grip force and enhancing muscle strength.

### OLB-1 and OLB-2 Significantly Reducing the Number of Rotations in APO-Induced 6-OHDA Parkinson's Disease Rats

After 3 weeks of modeling, the rotations of rats were recorded, the rats were induced to rotate, and behavioral changes of the rats were observed in a quiet and spacious environment. There is no rotation in a sham-operated group, and there is no significant difference between the groups injected with 6-OHDA, and there are about 180 rotations. After 2 weeks of treatment, the number of rotations increases slightly in the model group treated with normal saline; after 2 weeks of different dosages of OLB-1 and OLB-2, TMP and positive control drug L-dopa, the results are as follows: the treatment with different dosages of the OLB-1, OLB-2 and positive control levodopa (25 mg/kg) could effectively reduce the number of rotations in APO-induced 6-OHDA rats. Compared with the 6-OHDA model group, OLB-1 and OLB-2 treated rats showed a significant reduction in the number of rotations. Data were presented as mean±SEM; n=9-10 per group. One-way ANOVA and multiple range test were adopted to show differences between the two groups. *p<0.05, **p<0.01 vs. before 6-OHDA group.

As shown in FIG. 8, OLB-1 and OLB-2 had therapeutic effects on Parkinson's disease, significantly reducing the number of rotations.

The foregoing description is a general description of the present disclosure. Changes in form and substitution of equivalents may be made according to circumstances or actual needs. Although specific terms are used herein, these terms are intended to be descriptive rather than limiting. In addition, it should be understood that various changes and modifications may be made on the present disclosure by those skilled in the art after reading the content of the present disclosure, and these equivalent forms also fall within the scope defined by the appended claims of the present disclosure.

What is claimed is:

1. A pyrazine compound, a stereoisomer, a tautomer, and a pharmaceutically acceptable salt thereof, wherein the pyrazine compound is a compound of formula I: 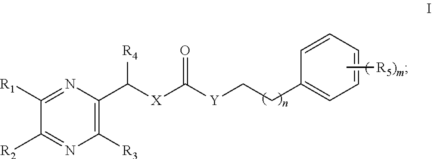
X and Y each are independently selected from the group consisting of O, S, Se, and NR₆; R₁, R₂, R₃, R₄, R₅, and R₆ each are independently selected from the group consisting of H, deuterium, halogen, hydroxyl, amino, carboxyl, acylamino, ester, alkyl, alkenyl, alkynyl, cycloalkyl, heterocyclyl, alkoxy, alkylcarboxyl, alkylester, -alkyl-OH, alkoxy, alkylamino, -alkyl-NH₂, -aryl, heteroaryl, carbonate, carbamate, -alkyl-acylamino, -aminocarboxylate, and a deuterated derivative thereof; and n is 0 to 6, m is 0 to 5.

2. A pyrazine compound, a stereoisomer, a tautomer, and a pharmaceutically acceptable salt thereof, wherein the pyrazine compound is a compound of formula I: 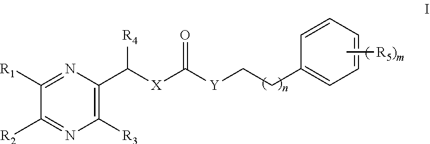
X and Y each are independently selected from the group consisting of O, S, Se, and NR₆; R₁, R₂, R₃, R₄, R₅, and R₆ each are independently selected from the group consisting of H, deuterium, halogen, hydroxyl, amino, carboxyl, acylamino, ester, substituted or unsubstituted alkyl, deuterated alkyl, substituted or unsubstituted alkenyl, substituted or unsubstituted alkynyl, substituted or unsubstituted cycloalkyl, substituted or unsubstituted heterocyclyl, substituted or unsubstituted alkoxy, substituted or unsubstituted alkylcarboxyl, substituted or unsubstituted alkylester, -substituted or unsubstituted alkyl-OH, substituted or unsubstituted alkoxy, substituted or unsubstituted alkylamino, -substituted or unsubstituted alkyl-NH₂, substituted or unsubstituted aryl, substituted or unsubstituted heterocyclic aryl, substituted or unsubstituted carbonate, substituted or unsubstituted carbamate, -substituted or unsubstituted alkyl-acylamino, -substituted or unsubstituted alkyl-aminocarboxylate, and a deuterated derivative thereof; and n is 0 to 6, m is 0 to 5.

3. The pyrazine compound, the stereoisomer, the tautomer, and the pharmaceutically acceptable salt thereof according to claim 1, wherein n is 0, 1, 2, 3, 4, 5, or 6; and m is 0, 1, 2, 3, 4, or 5.

4. The pyrazine compound, the stereoisomer, the tautomer, and the pharmaceutically acceptable salt thereof according to claim 1, wherein R₁, R₂, and R₃ each are selected from the group consisting of methyl and deuterated methyl.

5. The pyrazine compound, the stereoisomer, the tautomer, and the pharmaceutically acceptable salt thereof according to claim 1, wherein R₄ is selected from the group consisting of H and deuterium.

6. The pyrazine compound, the stereoisomer, the tautomer, and the pharmaceutically acceptable salt thereof according to claim 1, wherein the compound has a structure of formula II: 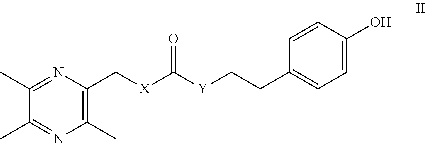
wherein X and Y each are selected from the group consisting of O, S, Se, and NR₆.

7. The pyrazine compound, the stereoisomer, the tautomer, and the pharmaceutically acceptable salt thereof according to claim 6, wherein the compound has a structure as follows:

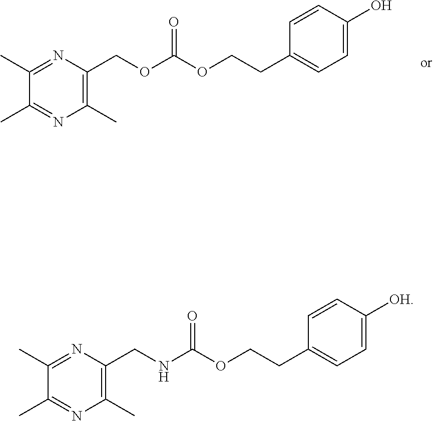 [SMILES: Cc1nc(C)c(COC(=O)OCCc2ccc(O)cc2)nc1C ; Cc1nc(C)c(CNC(=O)OCCc2ccc(O)cc2)nc1C]

8. The pyrazine compound, the stereoisomer, the tautomer, and the pharmaceutically acceptable salt thereof according to claim 1, wherein the pharmaceutically acceptable salt is obtained by reaction of the compound with hydrochloric acid, sulfuric acid, phosphoric acid, hydrobromic acid, nitric acid, salicylic acid, oxalic acid, benzoic acid, maleic acid, fumaric acid, citric acid, succinic acid, tartaric acid, C₁₋₆ aliphatic carboxylic acid, C₁₋₆ alkyl sulfonic acid, benzenesulfonic acid, p-toluenesulfonic acid, or camphorsulfonic acid.

9. A method of preparation of a compound, comprising the following steps:

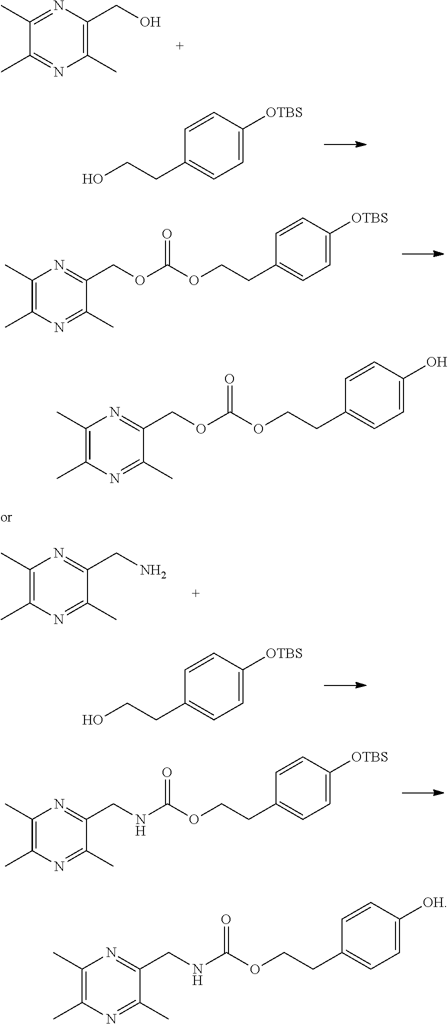

10. The method of preparation according to claim 9, comprising the following steps

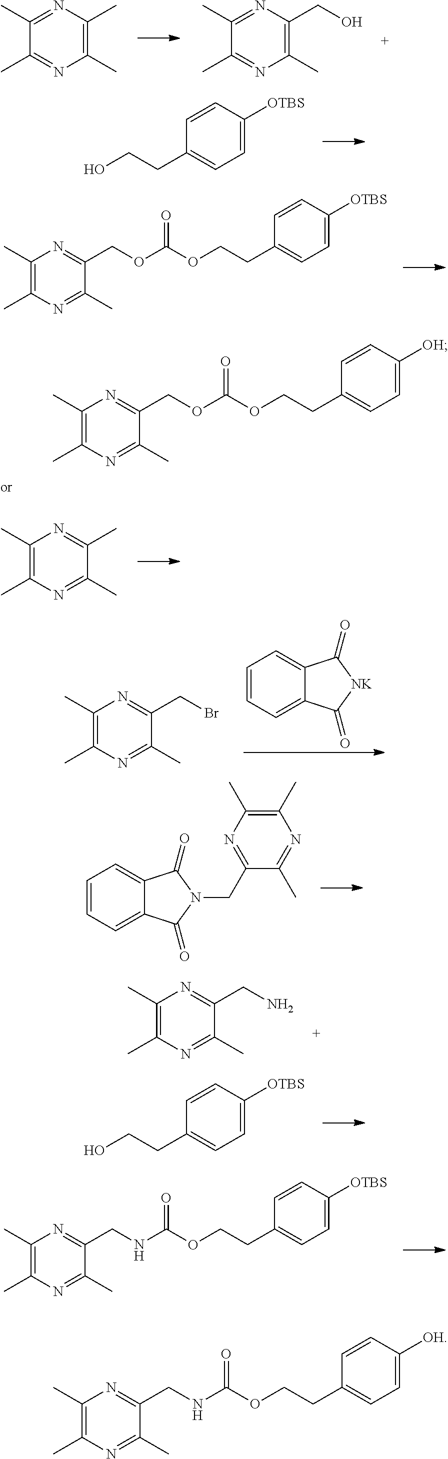

11. A compound, selected from the group consisting of the following compounds:

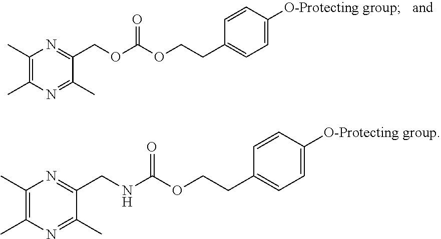

12. The compound according to claim 11, selected from the group consisting of the following compounds:

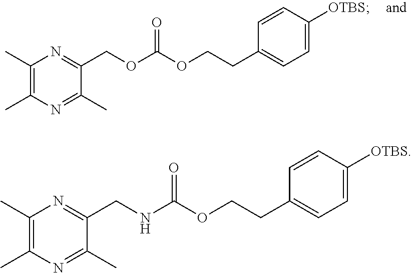 [SMILES: Cc1nc(C)c(COC(=O)OCCc2ccc(O[Si](C)(C)C(C)(C)C)cc2)nc1C ; Cc1nc(C)c(CNC(=O)OCCc2ccc(O[Si](C)(C)C(C)(C)C)cc2)nc1C]

13. A pharmaceutical composition, comprising a therapeutically effective amount of one or more of the pyrazine compound, the stereoisomer, the tautomer, and the pharmaceutically acceptable salt thereof according to claim 1.

14. A pharmaceutical composition, comprising a therapeutically effective amount of one or more of the compound according to claim 11.

15. The pharmaceutical composition according to claim 13, further comprising one or more pharmaceutically acceptable carriers or excipients.

16. The pharmaceutical composition according to claim 13, further comprising other therapeutic agents.

17. The pharmaceutical composition according to claim 13, wherein the pharmaceutical composition is capable of being prepared into a tablet, a granule, an injection, a gel, a pill, a capsule, a suppository, an implant, a nano preparation, and a powder for injection.

18. A method for treating a disease, comprising administering to a subject in need thereof a preparation of dosage unit of the pyrazine compound, the stereoisomer, the tautomer, and the pharmaceutically acceptable salt thereof according to claim 1, wherein the preparation contains a conventional pharmaceutically acceptable carrier, and wherein the disease is selected from the group consisting of a neurodegenerative disease (ND), an inflammation, an oxidative damage, a mitochondrial disorder-related disease, diabetes mellitus (DM), and a DM-related complication.

19. The use according to claim 18, wherein the ND comprises Alzheimer's disease, Parkinson's disease, Huntington's disease, frontotemporal dementia (FTD), vascular dementia, HIV-related dementia, multiple sclerosis, progressive lateral sclerosis, Friedreich's ataxia, neuropathic pain, and/or glaucoma.

20. The method according to claim 18, wherein the administering is performed by oral, injective, subcutaneous, respiratory, transdermal, parenteral, rectal, topical, intravenous, or intramuscular administration, or other means.

21. The method according to claim 18, wherein the pharmaceutically acceptable carrier comprises sugar, starch, cellulose, malt, gelatin, talc, and vegetable oil.
